(19) United States
(12) Patent Application Publication
(10) Pub. No.: US 2017/0081587 A1
(43) Pub. Date: Mar. 23, 2017

(54) LIQUID CRYSTAL COMPOSITION AND LIQUID CRYSTAL DISPLAY DEVICE INCLUDING THE SAME

(71) Applicant: Samsung Display Co., Ltd., Yongin-si (KR)

(72) Inventors: Jin Hyeong LEE, Hwaseong-si (KR); Sun Young KWON, Seoul (KR); Ji Hong BAE, Yongin-si (KR); Keun Chan OH, Cheonan-si (KR); Kyung Hee LEE, Suwon-si (KR); Seul Gee LEE, Seoul (KR)

(21) Appl. No.: 15/074,224

(22) Filed: Mar. 18, 2016

(30) Foreign Application Priority Data
Sep. 23, 2015 (KR) 10-2015-0134264

(51) Int. Cl. C09K 19/30; C07C 13/28; G02F 1/1368; C07C 43/168

(52) U.S. Cl. CPC C09K 19/30; C07C 43/168; C07C 13/28; G02F 1/1368; C07C 2101/16; C07C 2101/14

(57) ABSTRACT

A liquid crystal composition includes a first compound expressed by Chemical Formula I,

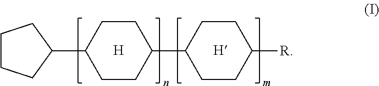

In Chemical Formula

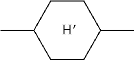

is at least one of a cyclohexyl group and phenyl group,

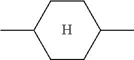

is at least one of a cyclohexyl group and a phenyl group, each of n and m is a natural number of 1 to 4, n+m is 2 to 5, and R is at least one of a C₁₋₁₀ alkyl group, a C₂₋₁₀ alkenyl group, and a C₁₋₁₀ alkoxy group.

### CROSS-REFERENCE TO RELATED APPLICATION

This application claims priority from and the benefit of Korean Patent Application No. 10-2015-0134264, filed on Sep. 23, 2015, which is hereby incorporated by reference for all purposes as if fully set forth herein.

### BACKGROUND

Field

Exemplary embodiments relate to a liquid crystal composition and a liquid crystal display device including the same.

Discussion of the Background

A liquid crystal display (LCD) is one of most widely used type of flat panel is display devices. Typically, an LCD includes two substrates having electric field generating electrodes, such as pixel electrodes, and a common electrode formed on one or both substrates. The LCD also includes a liquid crystal layer interposed between the two substrates. However, the versatility of LCDs is limited based on the slow response speed, low contrast, and high driving voltages.

The above information disclosed in this Background section is only for enhancement of understanding of the background of the inventive concept, and, therefore, it may contain information that does not form the prior art that is already known in this country to a person of ordinary skill in the art.

### SUMMARY

Exemplary embodiments provide a liquid crystal composition containing a novel liquid crystal compound having excellent low temperature stability and reliability.

Exemplary embodiments also provide a liquid crystal display device including a novel liquid crystal compound having excellent low temperature stability and reliability.

Additional aspects will be set forth in the detailed description which follows, and, in part, will be apparent from the disclosure, or may be learned by practice of the inventive concept.

An exemplary embodiment discloses a liquid crystal composition that includes a first compound expressed by Chemical Formula I,

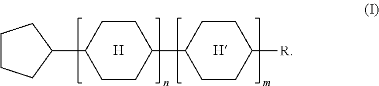

### In Chemical Formula I,

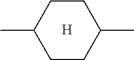

is at least one of a cyclohexyl group and a phenyl group,

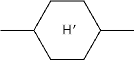

is at least one of a cyclohexyl group and a phenyl group, each of n and m is a is natural number of 1 to 4, n+m is 2 to 5, and R is at least one of a C₁₋₁₀ alkyl group, a C₂₋₁₀ alkenyl group, and a C₁₋₁₀ alkoxy group.

An exemplary embodiment also discloses a liquid crystal display device that includes a first display substrate comprising a thin film transistor, a second display substrate facing the first display substrate, and a liquid crystal layer including a first compound expressed by Chemical Formula I,

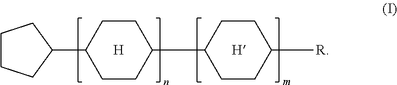

### In Chemical Formula I,

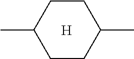

is at least one of a cyclohexyl group and a phenyl group,

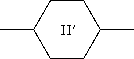

is at least one of a cyclohexyl group and a phenyl group, each of n and m is a natural number of 1 to 4, n+m is 2 to 5, and R is at least one of a C₁₋₁₀ alkyl group, a C₂₋₁₀ alkenyl group, and a C₁₋₁₀ alkoxy group.

The foregoing general description and the following detailed description are exemplary and explanatory and are intended to provide further explanation of the claimed subject matter.

### BRIEF DESCRIPTION OF THE DRAWINGS

The accompanying drawings, which are included to provide a further understanding of the inventive concept, and are incorporated in and constitute a part of this specification, illustrate exemplary embodiments of the inventive concept, and, together with the is description, serve to explain principles of the inventive concept.

FIG. 1 a schematic exploded perspective view of a liquid crystal display device according to an exemplary embodiment.

FIG. 2 is a schematic cross-sectional view of the liquid crystal display device of FIG. 1.

### DETAILED DESCRIPTION OF THE ILLUSTRATED EMBODIMENTS

In the following description, for the purposes of explanation, numerous specific details are set forth in order to provide a thorough understanding of various exemplary embodiments. It is apparent, however, that various exemplary embodiments may be practiced without these specific details or with one or more equivalent arrangements. In other instances, well-known structures and devices are shown in block diagram form in order to avoid unnecessarily obscuring various exemplary embodiments.

In the accompanying figures, the size and relative sizes of layers, films, panels, regions, etc., may be exaggerated for clarity and descriptive purposes. Also, like reference numerals denote like elements.

When an element or layer is referred to as being “on,” “connected to,” or “coupled to” another element or layer, it may be directly on, connected to, or coupled to the other element or layer or intervening elements or layers may be present. When, however, an element is or layer is referred to as being “directly on,” “directly connected to,” or “directly coupled to” another element or layer, there are no intervening elements or layers present. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various elements, components, regions, layers, and/or sections, these elements, components, regions, layers, and/or sections should not be limited by these terms. These terms are used to distinguish one element, component, region, layer, and/or section from another element, component, region, layer, and/or section. Thus, a first element, component, region, layer, and/or section discussed below could be termed a second element, component, region, layer, and/or section without departing from the teachings of the present disclosure.

Spatially relative terms, such as “beneath,” “below,” “lower,” “above,” “upper,” and the like, may be used herein for descriptive purposes, and, thereby, to describe one element or feature's relationship to another element(s) or feature(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted is accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof.

Various exemplary embodiments are described herein with reference to sectional illustrations that are schematic illustrations of idealized exemplary embodiments and/or intermediate structures. As such, variations from the shapes of the illustrations as a result, for example, of manufacturing techniques and/or tolerances, are to be expected. Thus, exemplary embodiments disclosed herein should not be construed as limited to the particular illustrated shapes of regions, but are to include deviations in shapes that result from, for instance, manufacturing. For example, an implanted region illustrated as a rectangle will, typically, have rounded or curved features and/or a gradient of implant concentration at its edges rather than a binary change from implanted to non-implanted region. Likewise, a buried region formed by implantation may result in some implantation in the region between the buried region and the surface through which the implantation takes place. Thus, the regions illustrated in the drawings are schematic in nature and their shapes are not intended to illustrate the actual shape of a region of a device and are not intended to be limiting.

The terms “disposed” and “disposing” generally mean “placed on” or “placing is on” but also include “formed on” and “forming on.” For example, a third layer disposed on a first layer is intended to include the third layer being formed separately and then placed on the first layer as well as the third layer being formed on the first layer. The third layer disposed on the first layer is not limited to being placed directly on or being formed directly on the first layer unless specifically stated. Thus, the third layer disposed on or formed on the first layer may have one or more intervening layers (e.g., a second layer) disposed or formed between the third layer and the first layer.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and will not be interpreted in an idealized or overly formal sense, unless expressly so defined herein.

LCDs need an increase in response speed and contrast as well as lower driving voltages to achieve higher versatility. This translates to an LCD that needs a liquid crystal layer that includes a liquid crystal composition having a lower rotational viscosity, higher chemical and physical stability, a higher liquid crystal phase-isotropic phase transition temperature, a low liquid crystal phase lower limit temperature, and a suitable elastic modulus. However, particular low viscosity liquid crystal compounds having an alkenyl group to improve response speed have drawbacks of poor low temperature stability. Thus, one or more exemplary embodiments below are directed to low viscosity liquid crystal compositions that exclude particular low viscosity liquid crystal compounds having an alkenyl group and improve the response speed of an LCD with low viscosity liquid crystal compounds without the poor low temperature stability.

Throughout the specification, each group of a liquid crystal compound will be referred to by the abbreviated nomenclature described below. Non-subscript integers refer to the number carbons in a straight carbon chain. For example, 2 in the formula 2P equates to an ethyl group (2) connected to a phenyl group (P) or ethylbenzene.

Table 1

TABLE 1

| | Nomen- | | Nomen- |
|---|---|---|---|
| Structure | clature | Structure | clature |
| 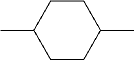 | C | 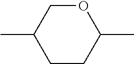 | N |
| 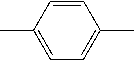 | P | 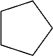 | Pt |
| 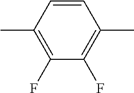 | A | 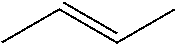 | V |
| 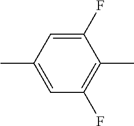 | K | 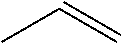 | V₁ |
| 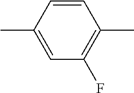 | L | 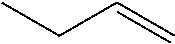 | V₂ |
| 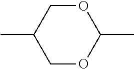 | D | —O— | O |

Exemplary embodiments are described hereinafter with reference to the accompanying drawings.

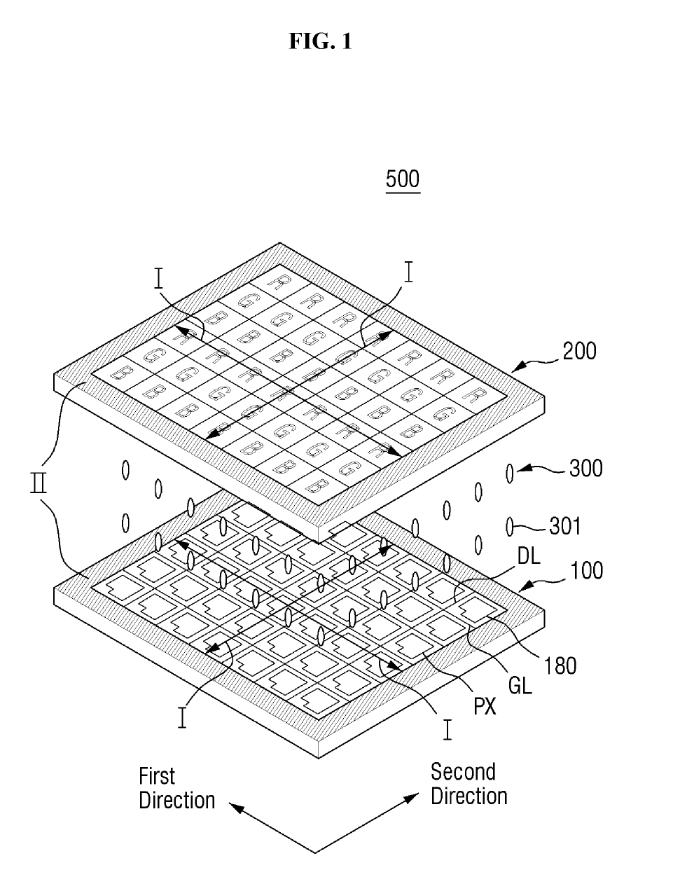

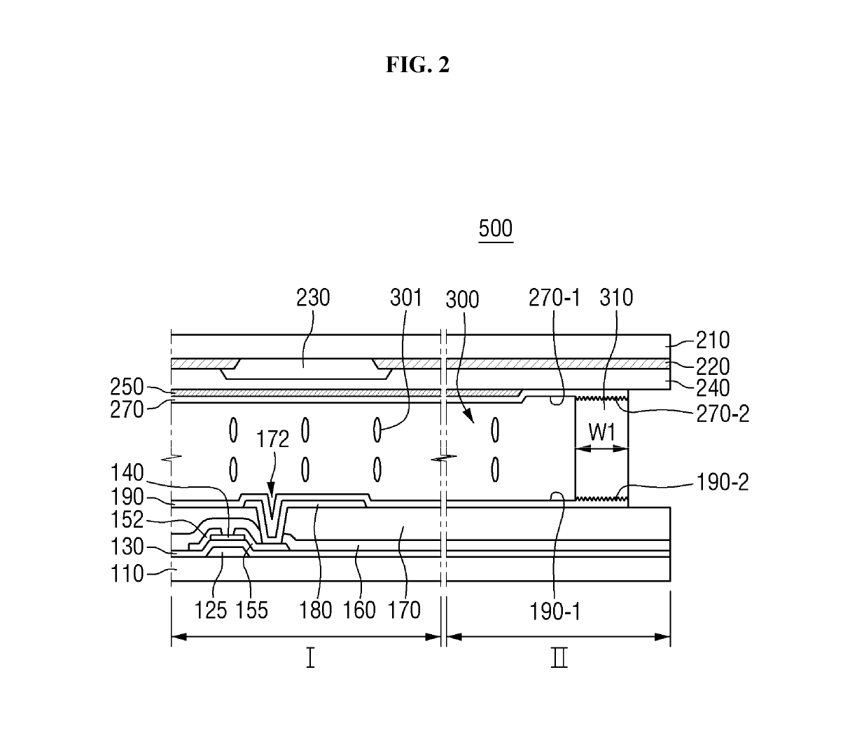

FIG. 1 is a schematic exploded perspective view of a liquid crystal display device according to an exemplary embodiment. FIG. 2 is a schematic cross-sectional view of the liquid crystal display device of FIG. 1.

Referring to FIG. 1 and FIG. 2, a liquid crystal display device 500 may include a first display substrate 100, a second display substrate 200 spaced apart from and facing the first display substrate 100, and a liquid crystal layer 300 interposed between the first display substrate 100 and the second display substrate 200. The first display substrate 100 and the second display substrate 200 may each include a display area I and a non-display area II. A plurality of pixels PXs may be arranged into a matrix form in the display area I.

A plurality of gate lines GLs may extend in a first direction and a plurality of data lines DLs may extend in a second direction vertical to the first direction in the display area I of is the first display substrate 100. A pixel electrode 180 may be disposed in each of the pixels defined by the gate lines GLs and data lines DLs.

The pixel electrode 180 may receive a data voltage through a thin film transistor that is a switching element. A gate electrode 125 that is a control terminal of the thin film transistor may be connected to the gate line GL. A source electrode 152 that is an input terminal of the thin film transistor may be connected to the data line DL. A drain electrode 155 that is an output terminal of the thin film transistor may be electrically connected to the pixel electrode 180 through a contact.

The thin film transistor may have a channel formed as a semiconductor layer 140. The semiconductor layer 140 may overlap the gate electrode 125. The source electrode 152 and the drain electrode 155 may be disposed on the semiconductor layer 140 and may be spaced apart from each other. The pixel electrode 180 may connect with a common electrode 250 such that they are configured to generate an electric field to control an alignment direction of a liquid crystal compound 301 in the liquid crystal layer 300 interposed between the pixel electrode 180 and the common electrode 250.

The non-display area II may be a periphery of the display area I and enclose the display area I. A driving unit may be disposed in the non-display area II of the first display substrate 100 to provide a gate driving signal, a data driving signal and the like to each pixel of the display area I.

A color filter 230 may be dispose in each pixel PX in the display area I of the second display substrate 200. The color filter 230 may include red, green, and blue color filters 230. The red, green, and blue color filters 230 may be arranged alternately with each other. For example, the red, green, and blue color filters 230 may be arranged alternately in only one direction (e.g., the second direction) as shown in FIG. 1. As an alternate example but not shown in FIG. 1, the red, green, and blue color filters 230 may be arranged alternately in two directions (e.g., the first and second direction). Regardless, a light blocking pattern 220 may be disposed at each boundary between the color filters 230. Furthermore, the light blocking pattern 220 may be disposed in the non-display area II of the second display substrate 200. The light blocking pattern 220 of the non-display area II may have a width wider than that of the light blocking pattern 220 formed at the boundary between the color filter 230. The common electrode 250 formed into an integrated body regardless of the pixel PX may be disposed on the whole surface of the display area I.

The first display substrate 100 and the second display substrate 200 may be bonded with each other by a seal line 310 that includes sealant or the like. The seal line 310 may be disposed at a periphery of the first display substrate 100 and the second display substrate 200. In particular, the seal line 310 may be disposed on the non-display area II. The seal line 310 may be disposed along a periphery of the display area I to enclose the display area I. Thus, the first display substrate 100 and the second display substrate 200 may be bonded with each other with a predetermined space defined therebetween by the seal line 310. The liquid crystal layer 300 may be provided in the defined space such that liquid crystal compounds 301 may be prevented from being leaked outwards.

The liquid crystal display device 500 will hereinafter be described in detail. The is first display substrate 100 may have a first substrate 110 as a base substrate. The first substrate 110 may have a display area I and a non-display area II. The first substrate 110 may include or be formed of a transparent insulation substrate such as glass and/or transparent plastic.

The gate line GL may include a conductive material. The gate electrode 125 may protrude from the gate line GL and may be disposed on the first substrate 110 in the display area I. Although not shown in the drawings, the gate line GL may extend to the non-display area II and form a gate pad (not shown) in the non-display area II. A gate insulation layer 130 may be disposed on and cover the gate line GL and the gate electrode 125. The gate insulation layer 130 may be disposed in display area I and the non-display area II.

The semiconductor layer 140 and an ohmic contact layer (not shown) may be disposed on the gate insulation layer 130 of the display area I. The source electrode 152 branched from the data line DL and the drain electrode 155 spaced apart from the source electrode 152 may be disposed on the semiconductor layer 140 and the ohmic contact layer. Although not shown in the drawings, the data line DL may extend to the non-display area II and form a data pad (not shown) in the non-display area II.

A passivation layer 160 (i.e., an insulation layer that may include at least one of a silicon nitride layer, a silicon oxide layer, and a silicon oxynitride layer) may be disposed on the source electrode 152 and the drain electrode 155. The passivation layer 160 is not limited to at least one of a silicon nitride layer, a silicon oxide layer, and a silicon oxynitride layer. The passivation layer may include any suitable layer or material.

An organic layer 170 may be disposed on the passivation layer 160. The organic layer 170 may include any suitable organic material. The passivation layer 160 and the organic layer 170 may be disposed in the non-display area II. In an alternate exemplary embodiment, the is passivation layer 160 is omitted from the first display substrate 100.

The pixel electrode 180 may be disposed in each pixel PX on the organic layer 170 in the display area I. The pixel electrode may include or may be formed of a conductive material. The pixel electrode 180 may be electrically connected to the drain electrode 155 through a contact hole 172. The contact hole 172 may penetrates through the organic layer 170 and the passivation layer 160 to expose the drain electrode 155. The pixel electrode 180 may include at least one of indium tin oxide, indium zinc oxide, indium oxide, zinc oxide, tin oxide, gallium oxide, titanium oxide, aluminum, silver, platinum, chrome, molybdenum, tantalum, niobium, zinc, and magnesium. The pixel electrode 180 may include an alloy of at least one of indium tin oxide, indium zinc oxide, indium oxide, zinc oxide, tin oxide, gallium oxide, titanium oxide, aluminum, silver, platinum, chrome, molybdenum, tantalum, niobium, zinc, and magnesium. The pixel electrode 180 may be a stacked film of one or more of the previously listed materials.

The second display substrate 200 will now be described. The second display substrate 200 may have a second substrate 210 as a base substrate. The second substrate 210 may include or may be formed of a transparent insulation substrate such as glass and/or a transparent plastic.

The light blocking pattern 220 may be disposed on the second substrate 210 in the display area I and the non-display area II. The light blocking pattern 220 may expose a portion of the second substrate 210 in the display area I.

The color filter 230 may be disposed on a portion of the light blocking pattern 220 and the second substrate 210 in the display area I. For example, the color filter 230 may be directly disposed on the exposed second substrate 210 and a portion of the light blocking pattern is 220 in the display area I. An overcoat layer 240 may be disposed on the color filter 230 and the light blocking pattern 220 in the display area I and the non-display area II.

The common electrode 250 may be disposed on the overcoat layer 240. The common electrode 250 may include at least one of indium tin oxide, indium zinc oxide, indium oxide, zinc oxide, tin oxide, gallium oxide, titanium oxide, aluminum, silver, platinum, chrome, molybdenum, tantalum, niobium, zinc, and magnesium. The common electrode 250 may include an alloy of at least one of indium tin oxide, indium zinc oxide, indium oxide, zinc oxide, tin oxide, gallium oxide, titanium oxide, aluminum, silver, platinum, chrome, molybdenum, tantalum, niobium, zinc, and magnesium. The common electrode 250 may include a stacked film of one or more of the previously listed materials.

The common electrode 250 may be disposed to cover the entire display area I. However, the common electrode 250 may have a slit (not shown) or an aperture (not shown) in the display area I. The common electrode 250 may be disposed in a part or portion of the non-display area II, but may not be disposed around an edge of the second display substrate 200 in order to expose the overcoat layer 240. The pixel electrode 180 of the first display substrate 100 and the common electrode 250 of the second display substrate 200 may face each other and both may be configured to form an electric field in the liquid crystal layer 300.

The first display substrate 100 and the second display substrate 200 may face each other with a predetermined cell gap maintained between the first display substrate 100 and the second display substrate 200. The liquid crystal layer 300 may be interposed between the first display substrate 100 and the second display substrate 200 in the display area I and the non-display area II.

A first liquid crystal alignment layer 190 may be disposed on the first substrate is 110 of the first display substrate 100 and a second liquid crystal alignment layer 270 may be disposed on the second substrate 210 of the second display substrate 200. The first liquid crystal alignment layer 190 may be disposed between the first display substrate 100 and the liquid crystal layer 300 in the display area I and non-display area II. The first liquid crystal alignment layer 190 may be disposed between the first display substrate 100 and the seal line 310 in the non-display area II. The second liquid crystal alignment layer 270 may be disposed between the second display substrate 200 and the liquid crystal layer 300 in the display area I and the non-display area II. The second liquid crystal alignment layer 270 may be disposed between the second display substrate 200 and the seal line 310 in the non-display area II. In a non-limiting example, the first and second liquid crystal alignment layers 190 and 270 may be polyimide-based liquid crystal alignment layers.

The liquid crystal layer 300 will hereinafter be described in detail. The liquid crystal layer 300 may include a liquid crystal composition with at least one first compound among the compounds expressed by Chemical Formula 1 below:

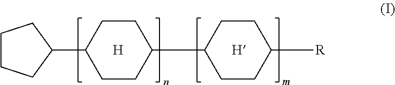

In Chemical Formula I,

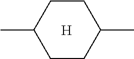

is a cyclohexyl group or a phenyl group,

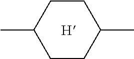

is a cyclohexyl group or a phenyl group, and R is at least one of a C₁₋₁₀ alkyl group, a C₂₋₁₀ alkenyl group, and a C₁₋₁₀alkoxy group.

In Chemical Formula I, each of n and m is a natural number of 1 to 4, and n+m is 2 to 5. When n+m is 1 (i.e., n+m is less than 2), that is, when only

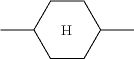

is a cyclohexyl group or a phenyl group, only

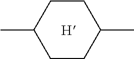

is a cyclohexyl group or a phenyl group, the phase transition temperature Tₙᵢ of the first compound is approximately −10° C. (see Table 2 below) causing a nematic phase temperature range to be too narrow. When n+m exceeds 5, the first compound may have a high rotational viscosity, causing degradation in response characteristics a liquid crystal display device.

The first compound may not have crystal precipitation at the temperature of −20° C. due to a low melting point of the first compound. Thus, the first compound may have excellent low temperature stability. Similarly, the first compound may have a phase transition temperature Tₙᵢ exceeding 100° C. Accordingly, the first compound may have a wide nematic phase temperature range (see Tables 2-9 below).

Table 2 below shows a measurement result of a phase transition temperature and a refractive index anisotropy of the compound expressed in the Chemical Formula A below. The phase transition temperature Tₙᵢ is measured in degrees Celsius. The refractive index anisotropy is the difference in the extraordinary refractive n_{c} index and the ordinary refractive index nₒ (i.e., Δn=n_{c}-nₒ).

TABLE 2

| 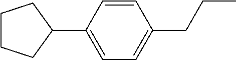 (A) | 3PPt |
|---|---|
| Phase transition temperature (Tₙᵢ) | −10° C. |
| refractive index anisotropy (Δn) or (nₑ-nₒ) | 0.08 |

When n is 2 or more, each

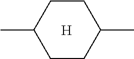

may be the same or different as each other. For example, when n is 2, both

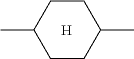

may be a cyclohexyl group, each

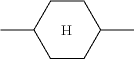

may be a phenyl group, or either one of the two

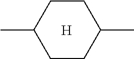

may be a cyclohexyl group and the other one of the two

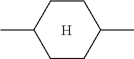

may be a phenyl group. As another example, when n is 3, all three

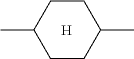

may be a cyclohexyl group, all of the three

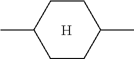

may be a phenyl group, or one of the three

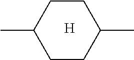

may be a cyclohexyl group, another one of the three

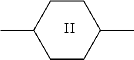

may be a phenyl group, and still another one of the three

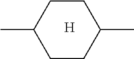

may be a cyclohexyl group or a phenyl group. As another example, when n is 4, all four

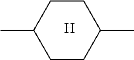

may be a cyclohexyl group all four

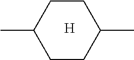

may be a phenyl group, or one of the four

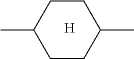

may be a cyclohexyl group, another one of the four

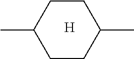

may be a phenyl group, still another one of the four

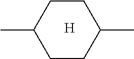

may be a cyclohexyl group or a phenyl group, and a still further one of four

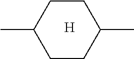

may be a cyclohexyl group or a phenyl group.

Similarly, when m is 2 or more, each

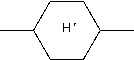

may be the same or different as each other. Since a person skilled in the art would easily understand an example in which m is 2, an example in which m is 3, and an example in which m is 4 with reference to the example in which n is 2, the example in which n is 3, and the example in which n is 4, descriptions of the examples in which m is 2, 3, and 4 will be omitted. Thus, Chemical Formula I may include each

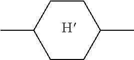

as a cyclohexyl group or a phenyl group in any combination as decribed above with respesct

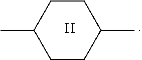

As described above, a first compound may include any compound of Chemical Formula I when n+m equals 2 to 5. Specifically, the first compound may be one of the compounds expressed by Chemical Formulas I-1, I-2, I-3, I-4, I-5, and I-6 below. The compounds expressed by Chemical Formulas I-1, I-2, I-3, I-4, I-5, and I-6 below may have one five-membered-ring and two six-membered-rings. One or more of the six-membered-rings may contain aromatic cyclic compounds. For example, Chemical Formulas I-3, I-4, I-5, and I-6 may contain one or more aromatic cyclic compounds (e.g., phenyl) as shown below.

Table 3 below shows a measurement result of dipole moment, dielectric anisotropy, rotational viscosity, and low temperature stability of the compound expressed by Chemical Formula I-1. The dielectric anisotropy is the difference between a parallel permittivity or dielectric constant ε∥ and a perpendicular permittivity or dielectric constant ε⊥ (i.e., Δε=ε∥−ε⊥). The rotational viscosity γ₁ is measured in miliPascal seconds.

TABLE 3

| | |
|---|---|
| 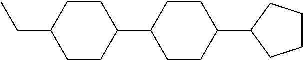 (I-1) | 2CCPt |
| Dipole moment | 0.0010 D |
| Dielectric anisotropy (Δε) or (ε_{∥}-ε_{⊥}) | −0.70 |
| Rotational viscosity (γ₁) | 92.0 mPa*s |
| Low temperature stability (−20° C.) | Satisfactory |

Table 4 below shows a measurement result of a dipole moment, a total energy, a phase transition temperature, a refractive index anisotropy, a dielectric anisotropy, rotational viscosity, and a low temperature stability of the compound expressed by Chemical Formula I-2.

TABLE 4

| | |
|---|---|
| 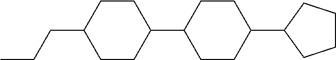 (I-2) | 3CCPt |
| Dipole moment | 0.0000 D |
| Total energy | 31.4477 kcal/mol |
| Phase transition temperature (Tni) | 129° C. |
| Refractive index anisotropy (Δn) or (nₑ-nₒ) | 0.0535 |
| Dielectric anisotropy (Δε) or (ε_{∥}-ε_{⊥}) | −0.8494 |
| Rotational viscosity (γ₁) | 98.9 mPa*s |
| Low temperature stability (−20° C.) | Satisfactory |

Table 5 below shows a measurement result of a dipole moment, a total energy, a phase transition temperature, a refractive index anisotropy, a dielectric anisotropy, and a rotational viscosity of the compound expressed by Chemical Formula I-3.

TABLE 5

| | |
|---|---|
| 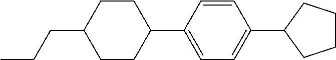 (I-3) | V₂CPPt |
| Dipole moment | 0.0279 D |
| Total energy | 20.1773 kcal/mol |
| Phase transition temperature (Tₙᵢ) | 130° C. |
| Refractive index anisotropy (Δn) or (nₑ-nₒ) | 0.1175 |
| Dielectric anisotropy (Δε) or (ε_{∥}-ε_{⊥}) | −0.7453 |
| Rotational viscosity (γ₁) | 95.9 mPa*s |

Table 6 below shows a measurement result of a dipole moment, a total energy, a phase transition temperature, a refractive index anisotropy, a dielectric anisotropy, and a rotational viscosity of the compound expressed by Chemical Formula I-4.

TABLE 6

| | |
|---|---|
| 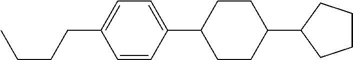 (I-4) | 4PCPt |
| Dipole moment | 0.0225 D |
| Total energy | 21.7689 |
| | kcal/mol |
| Phase transition temperature (Tₙᵢ) | 160° C. |
| Refractive index anisotropy (Δn) or (nₑ-nₒ) | 0.1069 |
| Dielectric anisotropy (Δε) or (ε_{∥}-ε_{⊥}) | −0.8907 |
| Rotational viscosity (γ₁) | 95.2 mPa*s |

Table 7 below shows a measurement result of a dipole moment, a total energy, a phase transition temperature, a refractive index anisotropy, a dielectric anisotropy, and a rotational viscosity of the compound expressed by Chemical Formula I-5.

TABLE 7

| | |
|---|---|
| 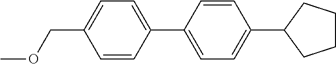 [SMILES: COCc1ccc(-c2ccc(C3CCCC3)cc2)cc1] (I-5) | 1O2PPPt |
| Dipole moment | 0.4715 D |
| Total energy | 21.4570 |
| | kcal/mol |
| Phase transition temperature (Tₙᵢ) | 117° C. |
| Refractive index anisotropy (Δn) or (nₑ-nₒ) | 0.1910 |
| Dielectric anisotropy (Δε) or (ε_{∥}-ε_{⊥}) | −1.4058 |
| Rotational viscosity (γ₁) | 91.8 mPa*s |

Table 8 below shows a measurement result of a phase transition temperature and a refractive index anisotropy of the compound expressed by Chemical Formula I-6.

TABLE 8

| 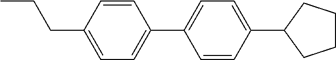 [SMILES: CCCc1ccc(-c2ccc(C3CCCC3)cc2)cc1] (I-6) | 3PPPt |
|---|---|
| Phase transition temperature (Tₙᵢ) | 130° C. |
| Refractive index anisotropy (Δn) or (nₑ-nₒ) | 0.08 |

The first compound may not be limited to the compounds expressed by the Chemical Formulas I-1, I-2, I-3, I-4, I-5, and I-6 above having one five-membered-ring and two six-membered-rings. The first compound may have one five-membered-ring and three six-membered-rings. One or more of the six-membered-rings may contain aromatic cyclic compounds. The compounds expressed by Chemical Formulas I-7, I-8, I-9, and I-10 below have one five-membered-ring and three six-membered-rings with Chemical Formulas I-8, I-9, and I-10 having a six-member-ring containing an aromatic cyclic compound.

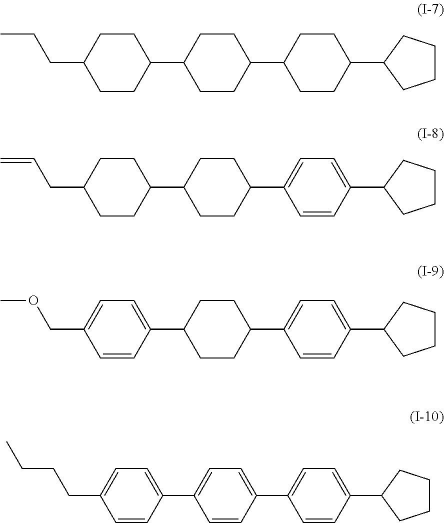

The liquid crystal composition may further include at least one second compound among the compounds expressed by Chemical Formulas II-1, II-2, II-3, II-4, II-5, II-6, II-7, and II-8 below.

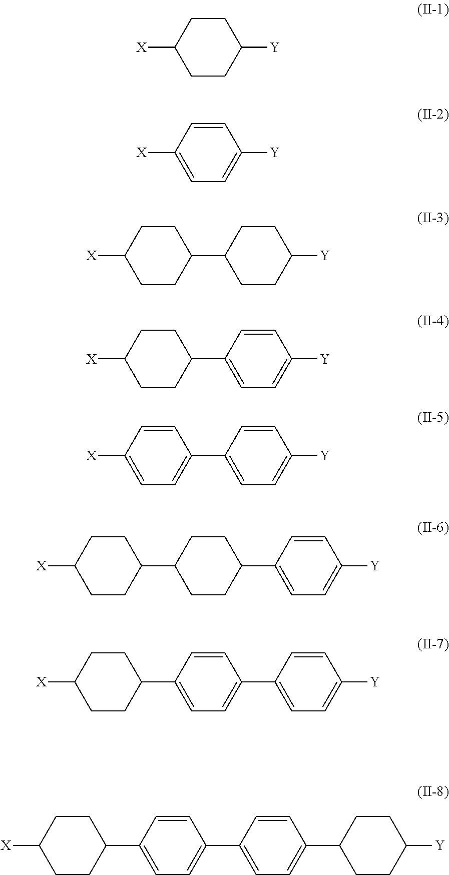

In the Chemical Formulas II-1, II-2, II-3, II-4, II-5, II-6, II-7, and II-8 above, each of X and Y, independent of one another, may be at least one of a C₁₋₁₀ alkyl group, a C₂₋₁₀ alkenyl group, a C₁₋₁₀ alkoxy group, a C₁₋₁₀ fluoroalkyl group, a C₂₋₁₀ fluoroalkenyl group, and a C₁₋₁₀ fluoroalkoxy group.

The sum of the content of the first compound and the content of the second compound may be 60 weight percent or less with respect to the total weight of the liquid crystal composition.

The liquid crystal composition may not include the compounds expressed by Chemical Formula 1 below among the compounds expressed by Chemical Formula II-3 above. In other words, a liquid crystal composition may include any second compound expressed by Chemical Formula II-3 except compounds that may be expressed by Chemical Formula 1 (not to be confused with Chemical Formula I above).

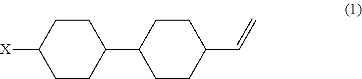

In the Chemical Formula 1, X is a C₁₋₁₀ alkyl group. The compounds expressed by Chemical Formula 1 may be a low viscosity liquid crystal compounds having a terminal alkenyl group. These compounds may be used in a liquid crystal composition to improve response characteristics, but may have poor low temperature stability. In other words, the compounds expressed by Chemical Formula 1 may be crystallized at the temperature of −20° C.

Examples of compounds expressed by Chemical Formula 1 may include the compound expressed by Chemical Formula I-1 below. Table 9 shows a measurement result of dipole moment, a dielectric anisotropy, a rotational viscosity, and a low temperature stability of is the compound expressed by Chemical Formula 1-1.

TABLE 9

| | |
|---|---|
| 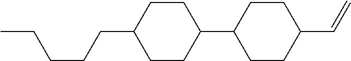 [SMILES: C=CC1CCC(C2CCC(CCCCC)CC2)CC1] (1-1) | 5CCV₁ |
| Dipole moment | 0.001 D |
| Dielectric anisotropy (Δε) or (ε_{∥}-ε_{⊥}) | −0.8000 |
| Rotational viscosity (γ₁) | 70.0 mPa*s |
| Low temperature stability (−20° C.) | Poor |

The liquid crystal composition may include a second compound expressed by Chemical Formula 2 below among compounds expressed by Chemical Formula II-3. The liquid crystal composition may include a second compound expressed by Chemical Formula 2 in an amount of 0 weight percent to 20 weight percent with respect to the total weight of the liquid crystal composition. In other words, the content of the second compound may include 20 weight percent or less of Chemical Formula 2 with respect to the total weight of the liquid crystal composition.

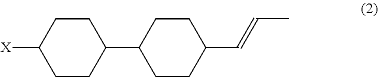

In exemplary embodiment, a liquid crystal composition of a high phase transition temperature may not include second compounds expressed by Chemical Formulas 1 and 2. In other words, the liquid crystal composition may exclude any second compound expressed by Chemical Formulas 1 and 2.

The liquid crystal composition and the liquid crystal layer may further include at least one third compound among compounds expressed by Chemical Formulas III-1, III-2, III-3, III-4, III-5, III-6, III-7, III-8, III-9, III-10, III-11, and III-12 below.

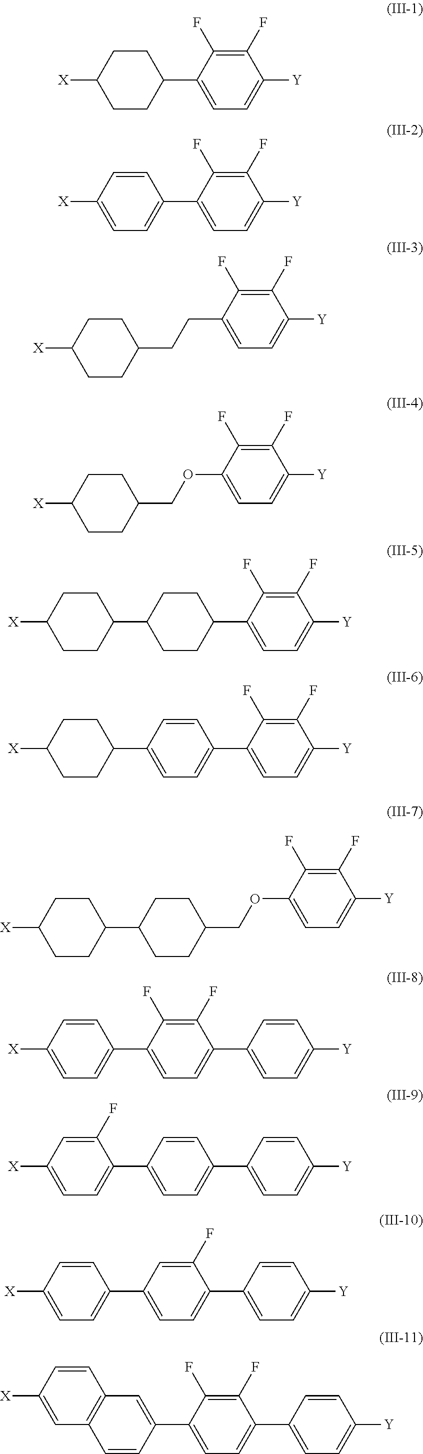

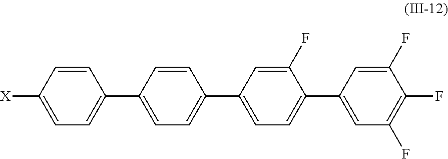

In the Chemical Formulas III-1, III-2, III-3, III-4, III-5, III-6, III-7, III-8, III-9, III-10, III-11, and III-12 above, each of X and Y may be, independently of one another, at least one of a C₁₋₆ alkyl group, a C₂₋₆ alkenyl group, a C₁₋₆ alkoxy group, a C₁₋₆ fluoroalkyl group, a C₂₋₆ fluoroalkenyl group, and a C₁₋₆fluoroalkoxy group.

Tables 10, 11, and 12 below show performance evaluation results of the liquid crystal compositions of preparation examples according to an exemplary embodiment and the liquid crystal composition of a comparative example.

### EXAMPLES

Referring to Tables 10, 11, and 12, the liquid crystal compositions according to an exemplary embodiment exhibits physical properties that are better than or equal to those of the liquid crystal composition of the comparative example. The liquid crystal compositions according to an exemplary embodiment have a reliability equal to or better than the liquid crystal composition of the comparative example. The liquid crystal compositions according to an exemplary embodiment have a low temperature stability better than the liquid crystal composition of the comparative example. Furthermore, the bend elastic constant K33 is given is for the exemplary embodiments and the comparative example in piconewtons pN below.

### Preparation Example 1

### Liquid Crystal Composition with High Reliability

TABLE 10

| | | Content | | | |
|---|---|---|---|---|---|
| | Liquid crystal | (weight | | | |
| | compound | percent) | Performance evaluation | | |
| 1 | 3CCV | 18 | Tₙᵢ | 74.5° | C. |
| 2 | 2CC3 | 10 | Δn = (nₑ − nₒ) | 0.109 | |
| 3 | V₂CPPt | 10 | Δε = (ε_{∥} − ε_{⊥}) | −3.0 | |
| 4 | 4PCPt | 5 | K33 | 15.8 | pN |
| 5 | 1O2PPPt | 5 | γ₁ | 99 | mPa*s |
| 6 | 3CAO4 | 5 | V₂CPPt = Chemical Formula I-3 | | |
| 7 | 5CAO2 | 4 | 4PCPt = Chemical Formula I-4 | | |
| 8 | 3CCAO2 | 12 | 1O2PPPt = Chemical Formula I-5 | | |
| 9 | 2CPAO2 | 5 | | | |
| 10 | 3CPAO2 | 10 | | | |
| 11 | 3PAO2 | 16 | | | |

### Preparation Example 2

### Liquid Crystal Composition with High Reliability

TABLE 11

| | | Content | | |
|---|---|---|---|---|
| | Liquid crystal | (weight | | |
| | compound | percent) | Performance evaluation | |
| 1 | 2CC3 | 20 | Δε = (ε_{∥} − ε_{⊥}) | −3.3 |
| 2 | 2CCPt | 15 | γ₁ | 136 mPa*s |
| 3 | 3CA02 | 20 | Low temperature | Excellent |
| | | | stability (−20° C.) | |
| 4 | 3CCA02 | 20 | Voltage holding ratio | 92.4% |
| | | | (VHR)_UV 10 J | |
| 5 | 3CPAO2 | 10 | 2CCPt = Chemical Formula I-1 | |
| 6 | 2PAP3 | 15 | | |

### Comparative Example

TABLE 12

| | | Content | | |
|---|---|---|---|---|
| | Liquid crystal | (weight | | |
| | compound | percent) | Performance evaluation | |
| 1 | 2CC3 | 20 | Δε = (ε_{∥} − ε_{⊥}) | −3.3 |
| 2 | 4CCV₁ | 15 | γ₁ | 133 mPa*s |
| 3 | 3CA02 | 20 | Low temperature | Poor |
| | | | stability (−20° C.) | |
| 4 | 3CCA02 | 20 | Voltage holding ratio | 89.2% |
| | | | (VHR)_UV 10 J | |
| 5 | 3CPAO2 | 10 | | |
| 6 | 2PAP3 | 15 | | |

Tables 13 and 14 below show performance evaluation results of the liquid crystal compositions of preparation examples according to an exemplary embodiment. Referring to Tables 13 and 14, the liquid crystal compositions according to an exemplary embodiment include liquid crystal composition including the first compound described above having high dielectric constant characteristics and high phase transition temperature characteristics.

### Preparation Example 2

### Liquid Crystal Composition with High Dielectric Constant

TABLE 13

| | | Content | | | |
|---|---|---|---|---|---|
| | Liquid crystal | (weight | | | |
| | compound | percent) | Performance evaluation | | |
| 1 | 3CCV | 15 | Tₙᵢ | 74.5° | C. |
| 2 | 2CC3 | 10 | Δn = (nₑ − nₒ) | 0.109 | |
| 3 | 3CCP1 | 3 | Δε = (ε_{∥} − ε_{⊥}) | −3.5 | |
| 4 | 4PCPt | 3 | K33 | 15.7 | pN |
| 5 | 1O2PPPt | 4 | γ₁ | 103 | mPa*s |
| 6 | 3CAO4 | 7 | 4PCPt = Chemical Formula I-3 | | |
| 7 | 5CAO2 | 6 | 1O2PPPt = Chemical Formula I-5 | | |
| 8 | 3CCAO1 | 13 | | | |
| 9 | 4CCAO2 | 8 | | | |
| 10 | 2CPAO2 | 5 | | | |
| 11 | 3CPAO2 | 5 | | | |
| 12 | 2PAP3 | 5 | | | |
| 13 | 3PAO2 | 16 | | | |

### Preparation Example 3

### Liquid Crystal Composition with High Phase Transition Temperature

TABLE 14

| | | Content | | | |
|---|---|---|---|---|---|
| | Liquid crystal | (weight | | | |
| | compound | percent) | Performance evaluation | | |
| 1 | 2CC3 | 15 | Tₙᵢ | 112° | C. |
| 2 | 3CC4 | 10 | Δn = (nₑ − nₒ) | 0.102 | |
| 3 | 3CCP1 | 3 | Δε = (ε_{∥} − ε_{⊥}) | −3.0 | |
| 4 | 3CCPt | 3 | K33 | 17.8 | pN |
| 5 | 4PCPt | 4 | γ₁ | 180 | mPa*s |
| 6 | 3CAO2 | 7 | 3CCPt = Chemical Formula I-2 | | |
| 7 | 3CCA1 | 6 | 4PCPt = Chemical Formula I-4 | | |
| 8 | 3CCAO2 | 13 | | | |
| 9 | 3CCAO3 | 8 | | | |
| 10 | 3CPAO2 | 5 | | | |
| 11 | 2PAP3 | 5 | | | |
| 12 | 4CPLP3 | 5 | | | |

While the present disclosure has been particularly shown and described with reference to exemplary embodiments thereof, it will be understood by those of ordinary skill in the art that various changes in implementation and detail may be made therein without departing from the spirit and scope of the following claims. The exemplary embodiments should be considered in a descriptive sense only and not for purposes of limitation.

What is claimed is:

1. A liquid crystal composition, comprising a first compound expressed by Chemical Formula I, 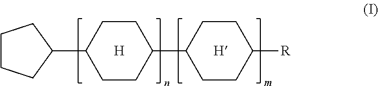
wherein, in Chemical Formula I, 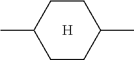
is at least one of a cyclohexyl group and a phenyl group, 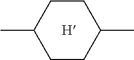
is at least one of a cyclohexyl group and a phenyl group, each of n and m is a natural number of 1 to 4, n+m is 2 to 5, and R is at least one of a C₁₋₁₀ alkyl group, a C₂₋₁₀ alkenyl group, and a C₁₋₁₀ alkoxy group.

2. The liquid crystal composition of claim 1, wherein the first compound is expressed by one of Chemical Formulas I-1, I-2, I-3, I-4, I-5, I-6, I-7, I-8, I-8, I-9, or I-10,

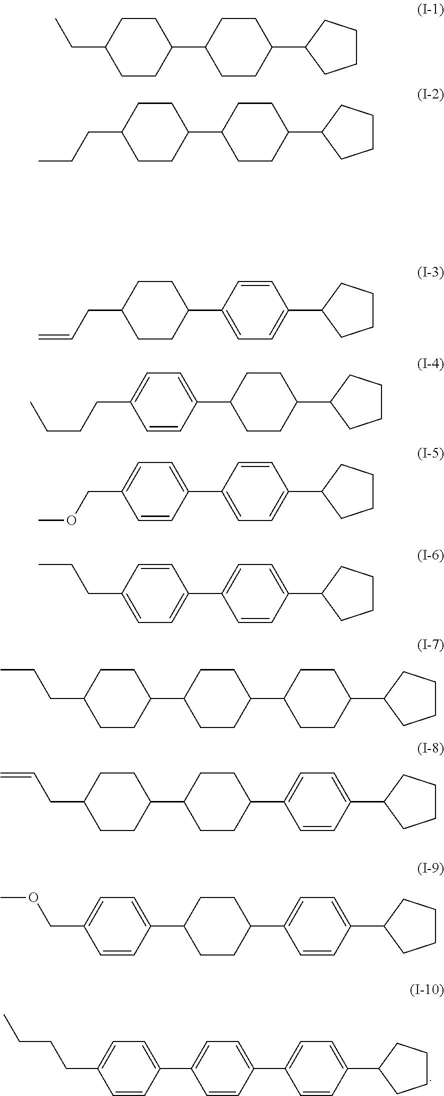

3. The liquid crystal composition of claim 1, further comprising a second compound expressed by one of Chemical Formulas II-1, II-2, II-3, II-4, II-5, II-6, II-7, or II-8, 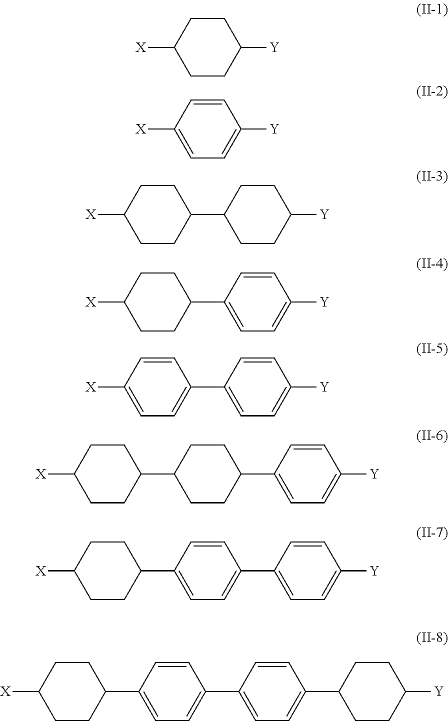
wherein, in Chemical Formulas II-1, 11-2, 11-3, 11-4, 11-5, 11-6, 11-7, and 11-8, each of X and Y are independently at least one of a C₁₋₁₀ alkyl group, a C₂₋₁₀ alkenyl group, a C₁₋₁₀ alkoxy group, a C₁₋₁₀ fluoroalkyl group, a C₂₋₁₀ fluoroalkenyl group, and a C₁₋₁₀ fluoroalkoxy group,
wherein the second compound excludes a compound expressed by Chemical Formula 1, 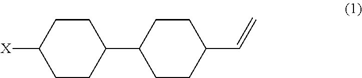
and wherein, in Chemical Formula 1, X is a C₁₋₁₀ alkyl group.

4. The liquid crystal composition of claim 3, wherein second compound further excludes a compound expressed by Chemical Formula 2, 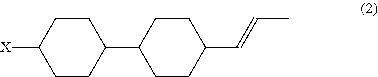
wherein, in Chemical Formula 2, X is a C₁₋₁₀ alkyl group.

5. The liquid crystal composition of claim 1, further comprising a second compound expressed by one of Chemical Formulas II-1, II-2, II-3, II-4, II-5, II-6, II-7, or II-8, wherein a sum of a content of the first compound and a content of the second compound is 60 weight percent or less with respect to a total weight of the liquid crystal composition, 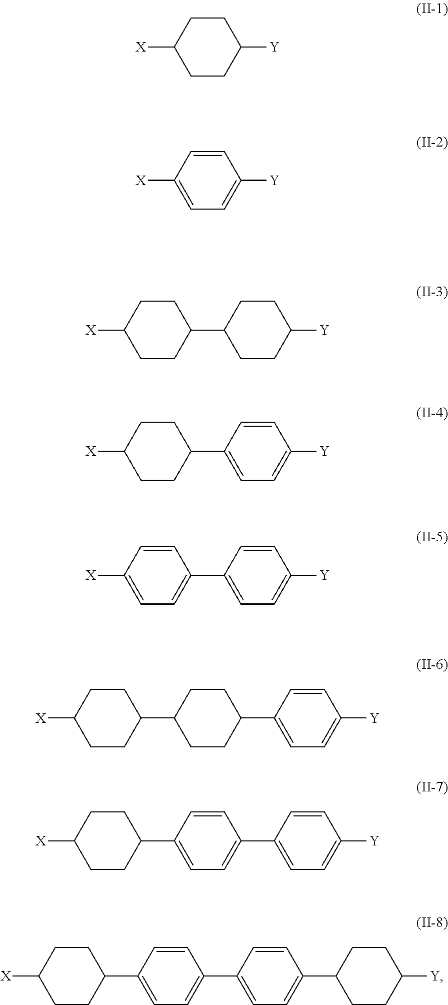
wherein, in chemical formulas II-1, II-2, II-3, II-4, II-5, II-6, II-7, and II-8, each of X and Y is independently at least one of a C₁₋₁₀ alkyl group, a C₂₋₁₀ alkenyl group, a C₁₋₁₀ alkoxy group, a C₁₋₁₀ fluoroalkyl group, a C₂₋₁₀ fluoroalkenyl group, and a C₁₋₁₀ fluoroalkoxy group,
wherein the content of the second compound expressed by Chemical Formula 2 is less than 20 weight percent with respect to the total weight of the liquid crystal composition, 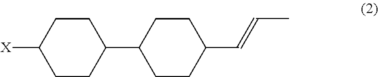
And wherein in Chemical Formula 2, X is a C₁₋₁₀ alkyl group.

6. The liquid crystal composition of claim 1, further comprising a third compound expressed by one of Chemical Formulas III-1, III-2, III-3, III-4, III-5, III-6, III-7, III-8, III-9, III-10, III-11, or III-12, 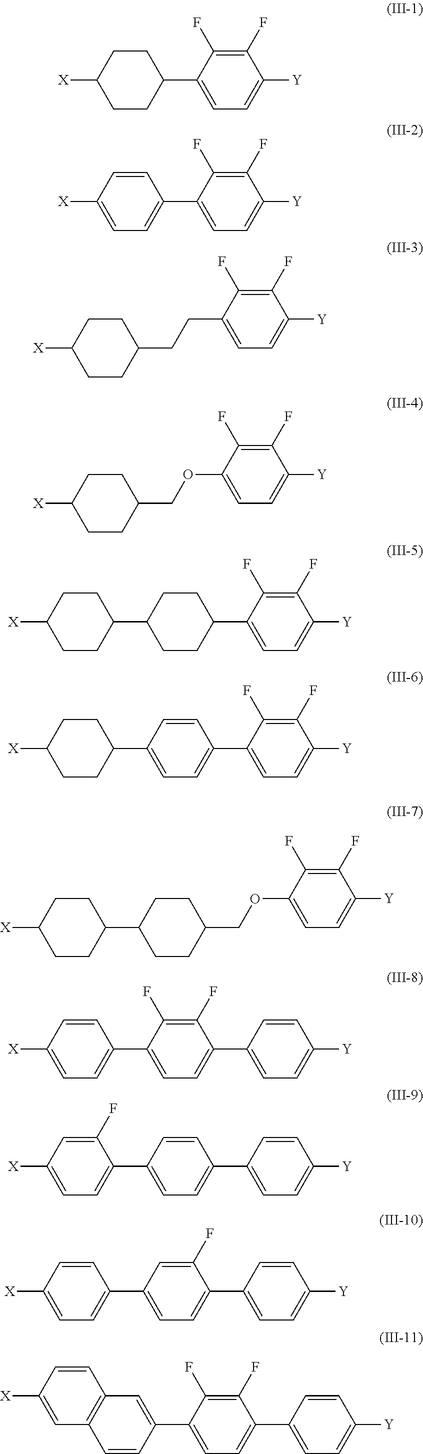 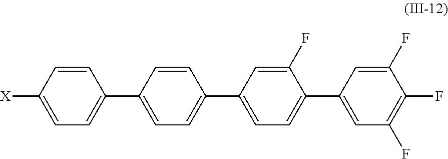
wherein, in Chemical Formulas III-1, III-2, III-3, III-4, III-5, III-6, III-7, III-8, III-9, III-10, III-11, and III-12, each of X and Y is independently at least one of a C₁₋₆ alkyl group, a C₂₋₆ alkenyl group, a C₁₋₆ alkoxy group, a C₁₋₆ fluoroalkyl group, a C₂₋₆ fluoroalkenyl group, and a C₁₋₆ fluoroalkoxy group.

7. A liquid crystal display device, comprising:
a first display substrate comprising a thin film transistor;
a second display substrate facing the first display substrate; and
a liquid crystal layer comprising a first compound s expressed by Chemical Formula I, 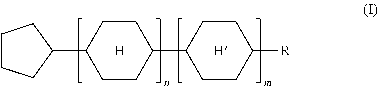
wherein, in Chemical Formula I, 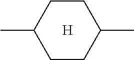
is at least one of a cyclohexyl group and a phenyl group, 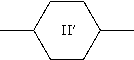
is at least one of a cyclohexyl group and a phenyl group, each of n and m is a natural number of 1 to 4, n+m is 2 to 5, and R is at least one of a C₁₋₁₀ alkyl group, a C₂₋₁₀ alkenyl group, and a C₁₋₁₀ alkoxy group.

8. The liquid crystal display device of claim 7, wherein the first compound is expressed by one of Chemical Formulas I-1, I-2, I-3, I-4, I-5, I-6, I-7, I-8, I-9, or I-10,

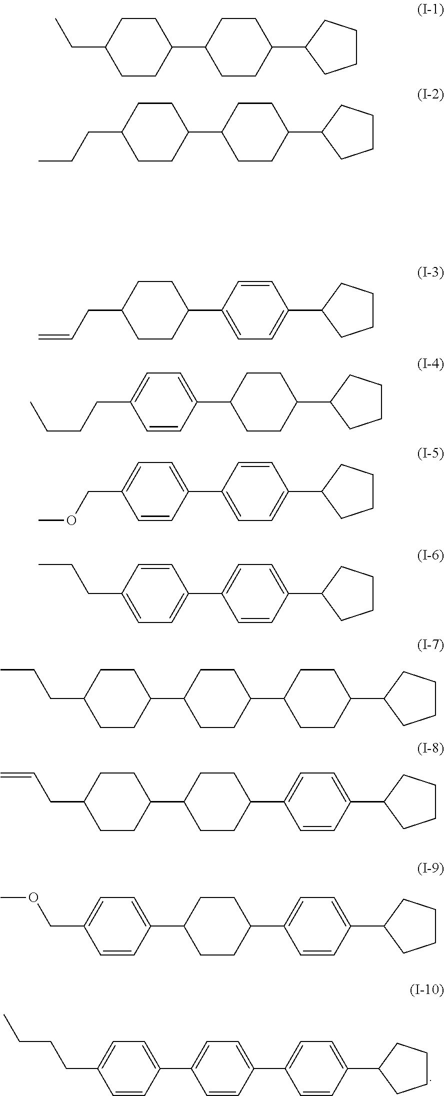

9. The liquid crystal display device of claim 7, wherein the liquid crystal layer further comprises a second compound expressed by one of Chemical Formulas II-1, II-2, II-3, II-4, II-5, II-6, II-7, or II-8, 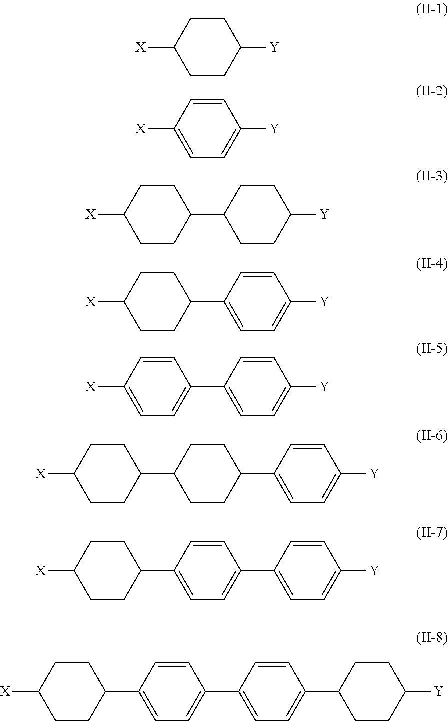
wherein, in Chemical Formulas II-1, II-2, II-3, II-4, II-5, II-6, II-7, and II-8, each of X and Y are independently at least one of a C₁₋₁₀ alkyl group, a C₂₋₁₀ alkenyl group, a C₁₋₁₀ alkoxy group, a C₁₋₁₀ fluoroalkyl group, a C₂₋₁₀ fluoroalkenyl group, and a C₁₋₁₀ fluoroalkoxy group,
wherein the second compound excludes Chemical Formula 1, 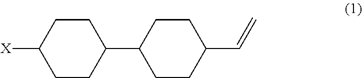
and wherein, in Chemical Formula 1, X is a C₁₋₁₀ alkyl group.

10. The liquid crystal display device of claim 9, wherein the second compound further excludes Chemical Formula 2, 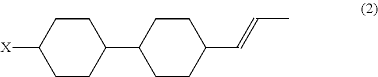
wherein, in Chemical Formula 2, X is a C₁₋₁₀ alkyl group.

11. The liquid crystal display device of claim 7, wherein the liquid crystal layer further comprises a second compound expressed by one of Chemical Formula II-1, II-2, II-3, II-4, II-5, II-6, II-7, or II-8
wherein a sum of a content of the first compound and a content of the second compound is 60 weight percent or less with respect to a total weight of a liquid crystal composition, 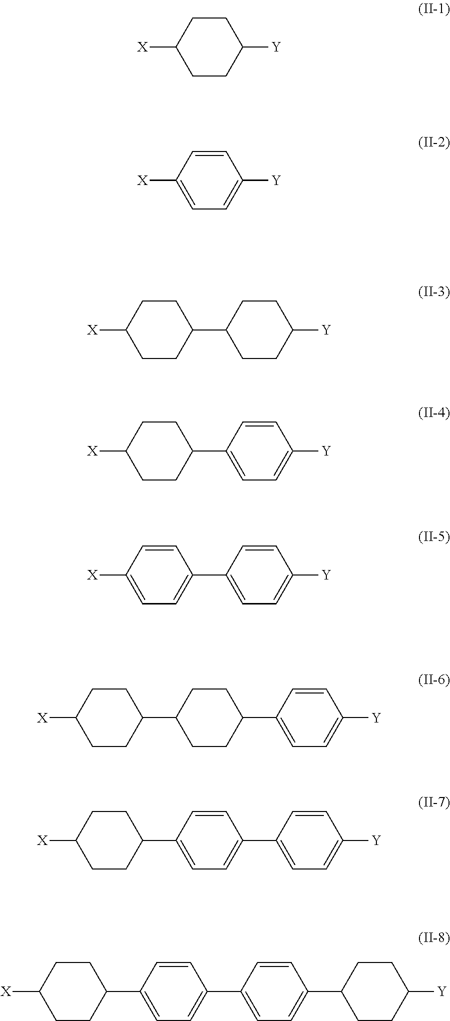
wherein, in Chemical Formulas II-1, II-2, II-3, II-4, II-5, I-6, II-7, and II-8, each of X and Y is independently at least one of a C₁₋₁₀ alkyl group, a C₂₋₁₀ alkenyl group, a C₁₋₁₀ alkoxy group, a C₁₋₁₀ fluoroalkyl group, a C₂₋₁₀ fluoroalkenyl group, and a C₁₋₁₀ fluoroalkoxy group,
wherein the content of the second compound expressed by Chemical Formula 2 is less than 20 weight percent with respect to the total weight of the liquid crystal composition: 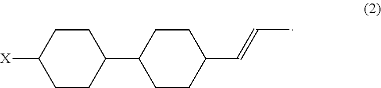
and wherein, in Chemical Formula 2, X is a C₁₋₁₀ alkyl group.

12. The liquid crystal display device of claim 7, wherein the liquid crystal layer further comprises a third compound expressed by one of Chemical Formulas III-1, III-2, III-4, III-4, III-5, III-6, III-7, III-8, III-9, III- 10, III-11, and III-12, 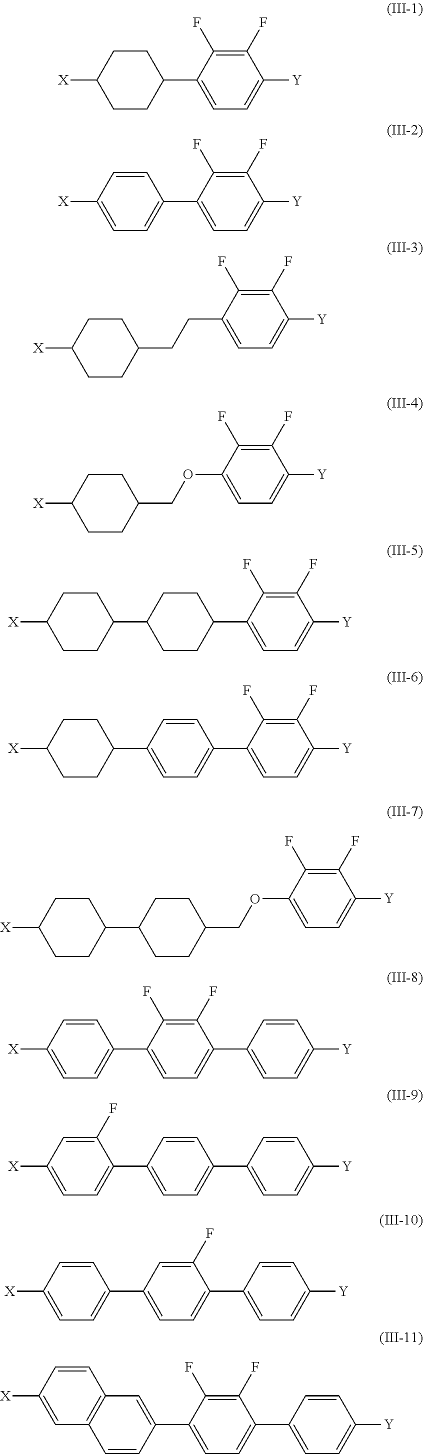 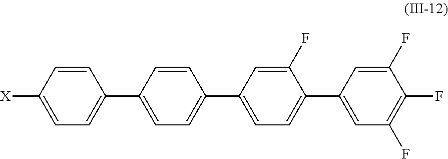
wherein, in Chemical Formulas III-1 to III-12, each of X and Y is independently at least one of a C₁₋₆ alkyl group, a C₂₋₆ alkenyl group, a C₁₋₆ alkoxy group, a C₁₋₆ fluoroalkyl group, a C₂₋₆ fluoroalkenyl group, and a C₁₋₆ fluoroalkoxy group.
